(19) United States
(12) Patent Application Publication
(10) Pub. No.: US 2025/0107774 A1
(43) Pub. Date: Apr. 3, 2025

(54) ULTRASOUND DIAGNOSTIC APPARATUS AND ULTRASOUND DIAGNOSTIC SYSTEM

(71) Applicant: FUJIFILM Corporation, Tokyo (JP)

(72) Inventors: Eiji KASAHARA, Tokyo (JP); Akira KUSAKABE, Tokyo (JP); Suguru ISHIGURO, Tokyo (JP); Koji WAKI, Tokyo (JP)

(73) Assignee: FUJIFILM Corporation, Tokyo (JP)

(21) Appl. No.: 18/895,356

(22) Filed: Sep. 24, 2024

(30) Foreign Application Priority Data
Sep. 28, 2023 (JP) 2023-167883

(51) Int. Cl. A61B 8/00; A61B 8/08

(52) U.S. Cl. CPC A61B 8/461; A61B 8/48

(57) ABSTRACT

An external image, which is an image generated through processing of an ultrasound image, and a display condition for displaying the external image are transferred from an external information processing apparatus to an ultrasound diagnostic apparatus. The display condition includes a designation of a specific display area in a selected specific screen layout. A display processing unit calculates an actual display position for displaying the external image based on the specific screen layout and the display condition to generate a composite image including the external image as an image to be displayed on a display.

### CROSS-REFERENCE TO RELATED APPLICATION

This application claims priority under 35 USC 119 from Japanese Patent Application No. 2023-167883, filed 28 Sep. 2023, the disclosure of which is incorporated by reference herein.

### BACKGROUND OF THE INVENTION

### 1. Field of the Invention

The present disclosure relates to an ultrasound diagnostic apparatus and an ultrasound diagnostic system, and particularly, to an image display technology.

### 2. Description of the Related Art

An ultrasound diagnostic system consisting of an ultrasound diagnostic apparatus and an external information processing apparatus is known. In such an ultrasound diagnostic system, an ultrasound image generated by the ultrasound diagnostic apparatus is processed by the external information processing apparatus. Specifically, the external information processing apparatus includes an application program for processing the ultrasound image (hereinafter, referred to as an external AP program or an external AP). The external AP program generates an image (hereinafter, referred to as an external image) representing a result of processing the ultrasound image.

In a case in which the external image is displayed on the external information processing apparatus, it is necessary to provide a display for the external information processing apparatus separately from a display of the ultrasound diagnostic apparatus, which may increase the bulk of the ultrasound diagnostic system. In addition, in that case, a user has to observe two displays, which may increase the burden on the user. It is desired to display the external image on the display of the ultrasound diagnostic apparatus together with the ultrasound image or instead of the ultrasound image.

JP2022-160214A and JP2022-167500A disclose an ultrasound diagnostic system consisting of an ultrasound diagnostic apparatus and an external information processing apparatus. JP2009-160233A discloses an ultrasound diagnostic apparatus that executes display control in accordance with a display layout. However, none of the above-described JP2022-160214A, JP2022-167500A, and JP2009-160233A disclose cooperation between the external information processing apparatus and the ultrasound diagnostic apparatus for the purpose of displaying the external image on the ultrasound diagnostic apparatus.

### SUMMARY OF THE INVENTION

In a case in which the external image generated by the external information processing apparatus is displayed on the display of the ultrasound diagnostic apparatus, the ultrasound diagnostic apparatus has to manage all pieces of information required for display processing of the external image for each external AP program in a case in which the ultrasound diagnostic apparatus determines all display conditions for the external image. In that case, in a case in which the external AP program is added or changed, it is necessary to add or change the information managed by the ultrasound diagnostic apparatus. That is, the ultrasound diagnostic system cannot be flexibly constructed. Since the external AP program naturally knows functions or properties of the image (external image) that the external AP program itself generates, it is rational to cause the external AP program to designate the basic display condition.

An object of the present disclosure is to realize a new mechanism for displaying an external image on a display of an ultrasound diagnostic apparatus.

According to the present disclosure, there is provided an ultrasound diagnostic apparatus including a processor and a display. The processor is configured to: select a specific screen layout from among a plurality of screen layouts; send an ultrasound image generated through transmission and reception of ultrasound waves to an external information processing apparatus; receive an external image, which is an image generated through processing of the ultrasound image, and a display condition, which is used to display the external image and which includes a designation of a specific display area in the specific screen layout, from the external information processing apparatus; and calculate an actual display position for displaying the external image based on the specific screen layout and the display condition to generate a composite image including the external image as an image to be displayed on the display.

According to the present disclosure, there is provided an ultrasound diagnostic system including an ultrasound diagnostic apparatus including a processor and a display, and an external information processing apparatus connected to the ultrasound diagnostic apparatus. The processor is configured to: select a specific screen layout from among a plurality of screen layouts; send an ultrasound image generated through transmission and reception of ultrasound waves to the external information processing apparatus; receive an external image, which is an image generated through processing of the ultrasound image, and a display condition, which is used to display the external image and which includes a designation of a specific display area in the specific screen layout, from the external information processing apparatus; and calculate an actual display position for displaying the external image based on the specific screen layout and the display condition to generate a composite image including the external image as an image to be displayed on the display.

According to the present disclosure, there is provided a program executed by an ultrasound diagnostic apparatus including a display, including: a function of selecting a specific screen layout from among a plurality of screen layouts; a function of sending an ultrasound image generated through transmission and reception of ultrasound waves to an external information processing apparatus; a function of receiving an external image, which is an image generated through processing of the ultrasound image, and a display condition, which is used to display the external image and which includes a designation of a specific display area in the specific screen layout, from the external information processing apparatus; and a function of calculating an actual display position for displaying the external image based on the specific screen layout and the display condition to generate a composite image including the external image as an image to be displayed on the display.

According to the present disclosure, it is possible to provide a new mechanism for displaying an external image on a display of an ultrasound diagnostic apparatus.

### BRIEF DESCRIPTION OF THE DRAWINGS

FIG. 1 is a block diagram showing an ultrasound diagnostic system according to an embodiment.

FIG. 2 is a diagram showing an example of a screen layout management table.

FIG. 3 is a diagram showing an example of a coordinate table.

FIG. 4 is a diagram showing an example of a screen layout.

FIG. 5 is a diagram showing an example of an external AP management table.

FIG. 6 is a diagram showing an example of a display condition.

FIG. 7 is a diagram showing an example of a code list.

FIG. 8 is a flowchart showing an example of an operation during system startup.

FIG. 9 is a flowchart showing an example of an operation during external image display.

FIG. 10 is a diagram showing a first example of a composite image including an external image.

FIG. 11 is a diagram showing a second example of the composite image including the external image.

FIG. 12 is a diagram showing a third example of the composite image including the external image.

FIG. 13 is a timing chart showing an operation example following a synchronous mode.

FIG. 14 is a timing chart showing an operation example following an asynchronous mode.

### DESCRIPTION OF THE PREFERRED EMBODIMENTS

Hereinafter, an embodiment will be described with reference to the drawings.

### (1) Outline of Embodiment

An ultrasound diagnostic apparatus according to the embodiment includes a processor and a display. The processor selects a specific screen layout from among a plurality of screen layouts. The processor sends an ultrasound image generated through transmission and reception of ultrasound waves to an external information processing apparatus, and receives an external image, which is an image generated through processing of the ultrasound image, and a display condition, which is used to display the external image and which includes a designation of a specific display area in the specific screen layout, from the external information processing apparatus. The processor calculates an actual display position for displaying the external image based on the specific screen layout and the display condition to generate a composite image including the external image as an image to be displayed on the display.

In the above configuration, the specific screen layout forms a common foundation for a designation of a printing condition by the external information processing apparatus and for display processing by the ultrasound diagnostic apparatus. That is, the external information processing apparatus designates the display condition on the premise of the specific screen layout. Meanwhile, the ultrasound diagnostic apparatus calculates the actual display position for displaying the external image in accordance with the display condition on the premise of the specific screen layout. With such a mechanism, in a case in which the external image is displayed on the display of the ultrasound diagnostic apparatus, it is possible to rationalize the cooperation between the external information processing apparatus and the ultrasound diagnostic apparatus. With the above configuration, even in a case in which the configuration of the external information processing apparatus is changed, it is not necessary to change the display processing in the ultrasound diagnostic apparatus, or the change amount of the display processing can be reduced.

The above processor may be configured with one or a plurality of physical processors. The specific screen layout may be selected by the ultrasound diagnostic apparatus based on the designation of the screen layout by the external information processing apparatus. The screen layout may be automatically designated by the ultrasound diagnostic apparatus, or the screen layout may be designated by the user. Information representing the selected specific screen layout may be provided from the ultrasound diagnostic apparatus to the external information processing apparatus as necessary. Each screen layout generally includes a plurality of display areas. The actual display position is display coordinates on a display coordinate system of the display.

In the embodiment, the above display condition further includes a designation of a display position in the specific display area. The display position is a position relative to the specific display area. With this configuration, a position in the specific display area, where the external image should be displayed, can be designated by the external information processing apparatus as necessary.

In the embodiment, the display condition further includes a designation of a display size of the external image. The display size is a size relative to the specific display area. The processor calculates an actual display size of the external image based on the designation of the display size. With this configuration, the display size or the display form of the external image can be designated by the external information processing apparatus as necessary.

In the embodiment, the display condition further includes a direct or indirect designation of the specific screen layout. The processor selects the specific screen layout based on the direct or indirect designation of the specific screen layout. With this configuration, the selection of the specific screen layout can be controlled by the external information processing apparatus. An attribute of the specific screen layout may be designated by the external information processing apparatus, instead of the specific screen layout itself. For example, the number of image display areas included in the screen layout may be designated as the attribute.

In the embodiment, the display condition further includes an update condition. The processor controls an update timing of the external image displayed on the display in accordance with the update condition. With this configuration, it is possible to designate a timing of updating the external image by the external information processing apparatus as necessary.

In the embodiment, the processor is configured to, in a case in which a synchronous mode is designated as the update condition, and a plurality of the external images are generated through a plurality of image processing operations by the external information processing apparatus, simultaneously update the plurality of external images displayed on the display. On the other hand, the processor is configured to, in a case in which an asynchronous mode is designated as the update condition, and the plurality of external images are generated through the plurality of image processing operations by the external information processing apparatus, independently update the plurality of external images displayed on the display.

An ultrasound diagnostic system according to the embodiment includes an ultrasound diagnostic apparatus including a processor and a display, and an external information processing apparatus connected to the ultrasound diagnostic apparatus. The processor selects a specific screen layout from among a plurality of screen layouts. The processor sends an ultrasound image generated through transmission and reception of ultrasound waves to the external information processing apparatus, and receives an external image, which is an image generated through processing of the ultrasound image, and a display condition, which is used to display the external image and which includes a designation of a specific display area in the specific screen layout, from the external information processing apparatus. The processor calculates the actual display position for displaying the external image based on the specific screen layout and the display condition to generate a composite image including the external image as an image to be displayed on the display.

A program according to the embodiment is a program executed by an ultrasound diagnostic apparatus including a display. The program according to the present embodiment includes: a selection function of selecting a specific screen layout from among a plurality of screen layouts; a transmission function of sending an ultrasound image generated through transmission and reception of ultrasound waves to an external information processing apparatus; a reception function of receiving an external image, which is an image generated through processing of the ultrasound image, and a display condition, which is used to display the external image and which includes a designation of a specific display area in the specific screen layout, from the external information processing apparatus; and a generation function of calculating an actual display position for displaying the external image based on the specific screen layout and the display condition to generate a composite image including the external image as an image to be displayed on the display.

A non-transitory storage medium according to the embodiment is a storage medium that stores a program for causing an ultrasound diagnostic apparatus as an information processing apparatus to execute a selection procedure, a transmission procedure, a reception procedure, and a generation procedure.

### (2) Details of Embodiment

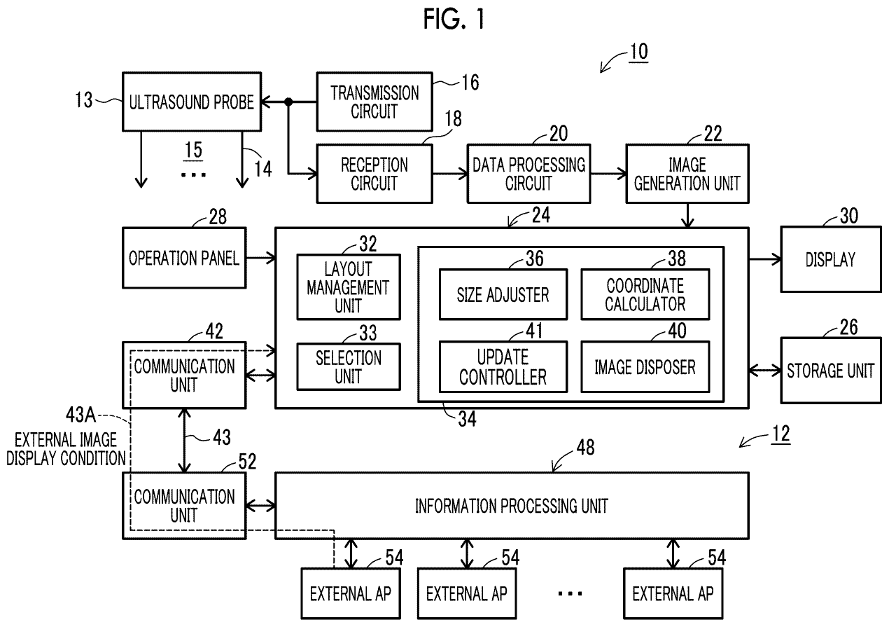

FIG. 1 shows an ultrasound diagnostic system according to the embodiment. The ultrasound diagnostic system is a medical system installed in a medical institution such as a hospital and used in an ultrasound examination of a living body. The ultrasound diagnostic system is composed of an ultrasound diagnostic apparatus 10 and an external information processing apparatus 12. The external information processing apparatus 12 is connected to the ultrasound diagnostic apparatus 10 via a dedicated cable 43. The external information processing apparatus 12 may be connected to the ultrasound diagnostic apparatus 10 via a network such as a local area network (LAN) or the Internet.

An ultrasound probe 13 is a device that transmits ultrasound waves into the living body and that receives reflected waves from the living body. The ultrasound probe 13 includes a transducer array consisting of a plurality of transducers. The transducer array forms an ultrasound beam 14. A beam scanning plane 15 is formed through electronic scanning of the ultrasound beam 14.

As an electronic scanning method of the ultrasound beam 14, an electronic linear scanning method, an electronic sector scanning method, and the like are known. A two-dimensional transducer array may be provided as the transducer array. Volume data may be acquired from a three-dimensional space in the living body by performing two-dimensional scanning with the ultrasound beam through the two-dimensional transducer array.

A transmission circuit 16 is an electronic circuit functioning as a transmission beam former and outputs a plurality of transmission signals to the transducer array in parallel during transmission. As a result, a transmission beam is formed.

A reception circuit 18 is an electronic circuit functioning as a reception beam former and applies phase addition to a plurality of reception signals output in parallel from the transducer array during reception, thereby generating reception beam data. With repetition of the electronic scanning of the ultrasound beam, a reception frame data sequence is output from the reception circuit 18. Each reception frame data is composed of a plurality of pieces of reception beam data that are arranged in an electronic scanning direction. Each reception beam data is composed of a plurality of pieces of echo data that are arranged in a depth direction.

A data processing circuit 20 is a circuit that processes each reception beam data. The data processing circuit 20 includes a detection circuit, a logarithmic transformation circuit, a filter, and the like. In addition, the data processing circuit 20 also includes a circuit that extracts and processes Doppler information included in each reception beam data.

An image generation unit 22 is an electronic circuit that generates a display frame data sequence (ultrasound image) based on the reception frame data sequence output from the data processing circuit 20. The image generation unit 22 includes a digital scan converter (DSC). The DSC has a coordinate transformation function, a pixel interpolation function, and the like. More specifically, for example, in a case in which a B-mode is selected, the image generation unit 22 generates a tomographic image data sequence as the display frame data sequence. In a case in which a color flow mapping (CFM) mode is selected, the image generation unit 22 generates the tomographic image data sequence and a blood flow image data sequence as the display frame data sequence. An operation mode other than the B-mode and the CFM mode may be selected.

A cine memory (not shown) is provided following the image generation unit 22. The cine memory has a ring buffer structure. The cine memory temporarily stores the tomographic image data sequence and/or the blood flow image data sequence. The image data sequence (the tomographic image data sequence and/or the blood flow image data sequence) read from the cine memory is sent to an information processing unit 24.

Hereinafter, both one image data and an image data sequence generated by the ultrasound diagnostic apparatus 10 will be simply referred to as an ultrasound image. Similarly, both one image data and an image data sequence generated by the external information processing apparatus 12 will be simply referred to as an external image.

A storage unit 26, an operation panel 28, and a display 30 are connected to the information processing unit 24, and a communication unit 42 is further connected to the information processing unit 24. The storage unit 26 is configured with, for example, a semiconductor memory. The operation panel 28 includes a plurality of switches, a track ball, a keyboard, and the like. The display 30 is configured with an organic EL display device, a liquid crystal display, or the like.

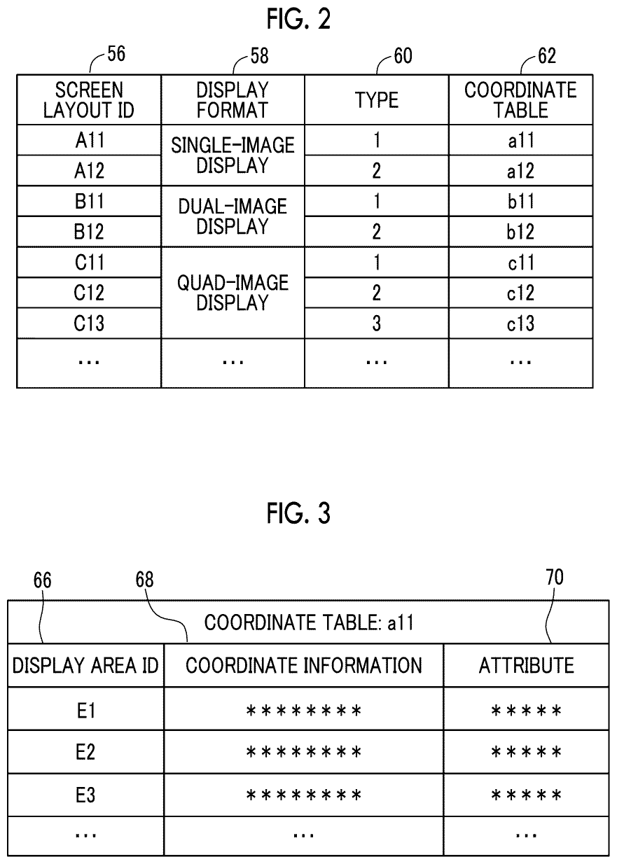

The storage unit 26 stores a screen layout management table for managing a plurality of screen layouts. The screen layout management table will be described below by using FIG. 2. In addition, the storage unit 26 stores a plurality of coordinate tables for implementing a plurality of screen layouts. The coordinate table will be described below by using FIG. 3. Further, the storage unit 26 stores an external AP management table for managing a plurality of external APs. The external AP management table will be described below by using FIG. 5.

The information processing unit 24 is configured with a processor that executes a program. The processor is specifically a central processing unit (CPU). The information processing unit 24 functions as a control unit that controls operations of a plurality of elements constituting the ultrasound diagnostic apparatus 10. In addition, the information processing unit 24 functions as a calculation unit that executes various calculations and a processing unit that executes various kinds of processing.

In FIG. 1, a plurality of representative functions exerted by the information processing unit 24 are represented by a plurality of blocks. Specifically, the information processing unit 24 includes a layout management unit 32, a selection unit 33, and a display processing unit 34.

The layout management unit 32 manages a plurality of selectable screen layouts. Each screen layout includes a plurality of display areas. Each display area is, for example, an image display area, an information display area, a message display area, or a state display area. However, these area types are not strict but are for convenience.

The selection unit 33 selects a specific screen layout from among the plurality of screen layouts based on the designation of the screen layout by the external information processing apparatus 12, the designation of the screen layout by the ultrasound diagnostic apparatus 10, or the designation of the screen layout by the user. The selection unit 33 changes the screen layout as necessary. For example, in a case in which a plurality of external images are simultaneously displayed, a specific screen layout that is suitable for all the plurality of external images is selected.

The display processing unit 34 generates a composite image in accordance with the selected specific screen layout. The composite image is an image to be displayed on a screen of the display 30 and includes, for example, the ultrasound image generated by the image generation unit 22 and the image (external image) generated by the external information processing apparatus 12. In addition, the composite image may include text information, a thumbnail image sequence, an icon sequence, and the like. The display processing unit 34 will be described in detail below.

The information processing unit 24 transmits the ultrasound image to the external information processing apparatus through the communication unit 42 and receives the external image and the display condition sent from the external information processing apparatus through the communication unit 42. A streaming technology may be used for the transfer of the image data sequence. The reception frame data or the reception frame data sequence before the coordinate transformation may be transferred from the ultrasound diagnostic apparatus 10 to the external information processing apparatus 12.

The external information processing apparatus 12 is configured with, for example, a computer. The external information processing apparatus 12 includes an information processing unit 48 and a communication unit 52. The information processing unit 48 includes a processor that executes a program. The processor is, for example, a CPU. The program includes a plurality of AP programs (a plurality of external APs) 54. Each external AP 54 is an image processing unit that processes the ultrasound image to generate an image (that is, an external image) indicating a processing result of the ultrasound image. One or a plurality of external APs 54 selected by the user or automatically from among the plurality of external APs 54 operate.

The external image and the display condition are transferred from the external AP 54 in operation to the information processing unit 24 via the communication unit 52 and the communication unit 42 (refer to reference numeral 43A). The display condition includes a designation of a specific display area in the specific screen layout, a designation of a display position in the specific display area, and an update condition, as will be described below. The display condition may also include a direct or indirect designation of the screen layout. The designation of the display position may also include a designation of a display size.

The plurality of external APs 54 include, for example, an AP program for detecting a lesion part in a tomographic image, an AP program for performing evaluation analysis on the detected lesion part, an AP program for analyzing a blood flow image or tissue movements, an AP program for measuring a size or an area of a tissue, an AP program for constructing a three-dimensional image, and the like.

In the ultrasound diagnostic apparatus 10, the display processing unit 34 processes the external image sent from the external information processing apparatus 12 in accordance with the display condition sent from the external information processing apparatus 12 on the premise of the selected specific screen layout. The display processing unit 34 includes a size adjuster 36, a coordinate calculator 38, an image disposer 40, and an update controller 41.

The size adjuster 36 calculates the actual display size of the external image based on the designation of the specific display area. More specifically, the size adjuster 36 calculates the actual display size, for example, such that the external image fits within the specific display area. The actual display size is a display size on the display coordinate system of the display 30. In a case in which the display condition includes the designation of the display size, the size adjuster 36 adjusts the actual display size of the external image in accordance with the designation of the display size. The actual display size is specifically defined by a coordinate range in a horizontal direction and a coordinate range in a vertical direction.

The coordinate calculator 38 calculates the actual display position for displaying the external image based on the designation of the display position in the specific display area. The actual display position is display coordinates on the display coordinate system of the display 30. More specifically, the coordinate calculator 38 calculates the display coordinates of each pixel constituting the external image. However, the display coordinates of one representative point in the external image may be calculated, or the display coordinates of a plurality of representative points in the external image may be calculated. The actual display position may be calculated after the calculation of the actual display size, the actual display position may be calculated before the calculation of the actual display size, or the actual display size and the actual display position may be simultaneously calculated.

The image disposer 40 disposes the external image having the calculated actual display size at the calculated actual display position on the display coordinate system. In accordance with the selected specific screen layout, the ultrasound image generated by the ultrasound diagnostic apparatus or other information (text information, a thumbnail image sequence, an icon sequence, and the like) is also disposed on the display coordinate system. Consequently, the composite image including the external image is generated. The composite image is displayed on the display 30. A composite image including a plurality of external images generated in parallel may be displayed. In a case in which important information is hidden in disposing the external image, the display position of the external image may be changed such that such a problem does not occur.

The update controller 41 controls the update timing of the external image displayed on the display 30 in accordance with the update condition included in the display condition. This will be described in detail below.

FIG. 2 shows an example of the screen layout management table. A display format 58, a type 60, and a coordinate table 62 are managed for each screen layout ID 56. The display format 58 and the type 60 correspond to the attribute of the screen layout. Examples of the display format 58 include single-image display, dual-image display, and quad-image display. In a case in which a plurality of screen layouts belong to one display format 58, the screen layouts are identified by types. The coordinate table 62 includes coordinate information of each display area constituting the screen layout. In managing the plurality of screen layouts, a table other than the screen layout management table shown in FIG. 2 may be constructed.

FIG. 3 shows an example of the coordinate table. Each individual screen layout includes a plurality of display areas. On the coordinate table, coordinate information 68 and an attribute 70 are managed for each display area ID 66. The coordinate information 68 is information for specifying coordinates of the display area in the display coordinate system, and the attribute 70 is information indicating the type of the display area. Examples of the display area include an image display area, an information display area, a message display area, and a state display area.

The coordinate table may have a structure other than the structure shown in FIG. 3.

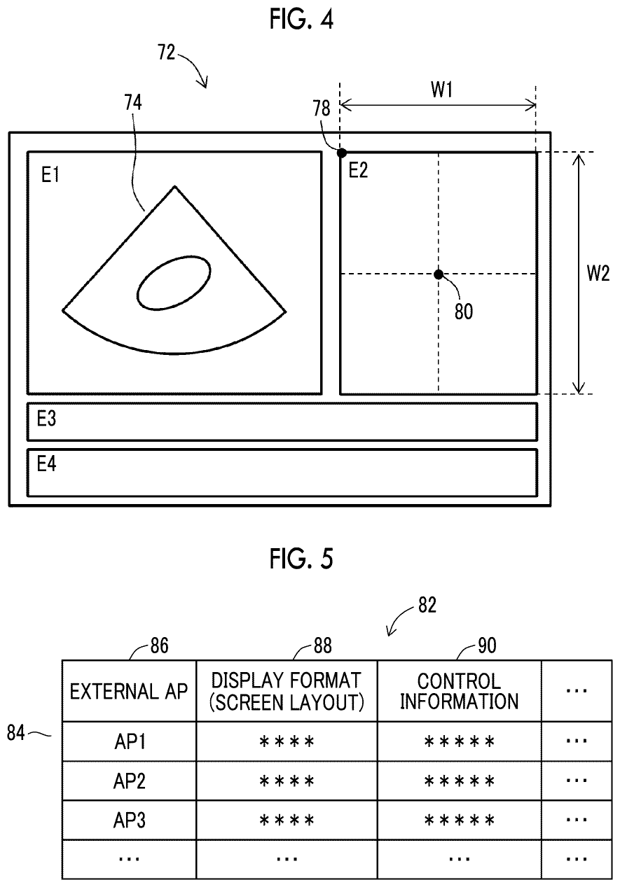

FIG. 4 shows an example of the screen layout. A screen layout 72 shown in FIG. 4 includes a plurality of display areas E1 to E4. The display area E1 is, for example, an image display area for displaying the ultrasound image. The display area E2 is, for example, an image display area for displaying the external image. The display area E2 has a width W1 in the horizontal direction and has a width W2 in the vertical direction. These widths W1 and W2 change depending on a display magnification or the like. A plurality of representative points are defined in the display area E2. The plurality of representative points include, for example, an upper left corner point 78 and a center point 80. The external image is disposed with any one of the representative points as a reference. For example, the text information, the icon sequence, and the like are displayed in the display area E3 and the display area E4.

FIG. 5 shows an example of the external AP management table. An external AP management table 82 includes a plurality of records 84 corresponding to the plurality of APs. Each record 84 includes information 86 for identifying the external AP, a display format 88 for displaying the image (external image) generated by the external AP, control information 90 for controlling the external AP, and the like. Information for specifying the screen layout may be managed instead of or in addition to the display format 88. The control information 90 is referred to, for example, in a case in which an external AP selection image is generated, and is referred to in a case in which an instruction or a command is sent to the external AP. In the embodiment, there is no need to refer to the control information 90 during the display processing of the external image.

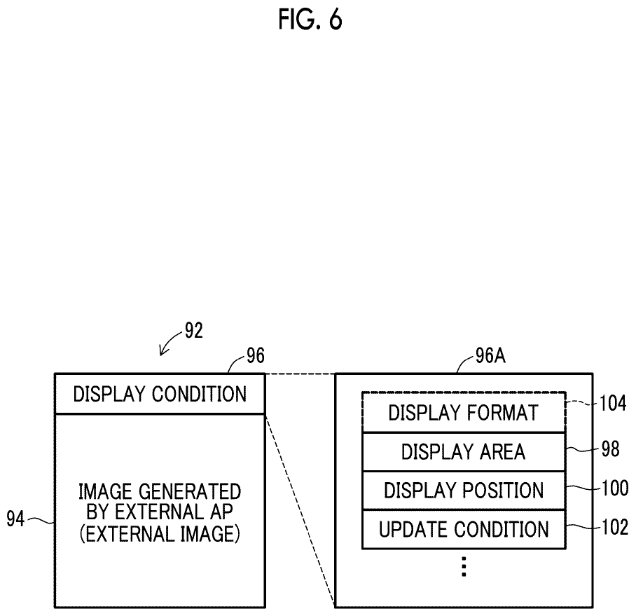

FIG. 6 shows an example of the information transferred from the external information processing apparatus to the ultrasound diagnostic apparatus. Shown information 92 includes an external image 94 and a display condition 96. The display condition 96 may be added to each individual external image 94, or the display condition may be added to an external image sequence.

Reference numeral 96A indicates contents of the display condition 96. The display condition 96 includes a display area designation 98, a display position designation 100, and an update condition 102 on the premise of the specific screen layout. The designated display area is any display area in the specific screen layout. The designated display position is a position relative to the specific display area. The display position designation 100 may include the designation of the display size in some cases. The designated display size is a size relative to the specific display area. The display position designation 100 is actually a designation of a code, which will be described below.

The update condition 102 means a mode for updating the external image. As will be described below, in the present embodiment, any of the synchronous mode or the asynchronous mode can be selected as the update condition 102. In a case in which the synchronous mode is selected, the plurality of external images displayed in parallel are simultaneously updated. In a case in which the asynchronous mode is selected, the plurality of external images displayed in parallel are independently updated.

A display format 104 may be included in the display condition 96. In that case, the selection unit in the ultrasound diagnostic apparatus may select the specific screen layout based on the display format 104. Information for specifying the screen layout may be included in the display condition 96. As described above, in a case in which the information for directly or indirectly specifying the screen layout is included in the display condition 96, the screen layout that is suitable for the external AP in operation is naturally selected.

In a case in which the plurality of external APs are operated in parallel, the display condition is provided from the external information processing apparatus to the ultrasound diagnostic apparatus for each external image. In that case, a screen layout having the largest number of image display areas may be selected from among the plurality of screen layouts that are suitable for a plurality of display conditions. That is, a screen layout that allows for the display of the plurality of external images may be selected.

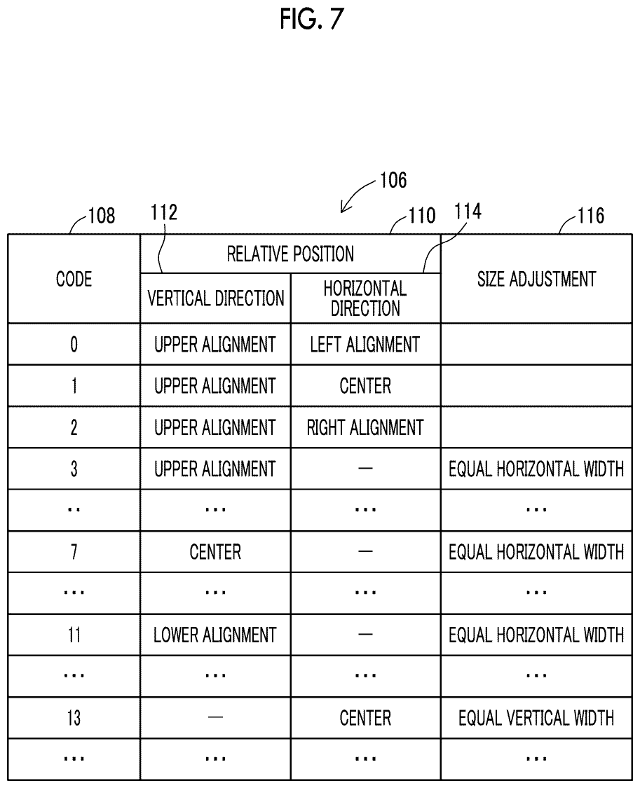

FIG. 7 shows an example of the code list. A shown code list 106 includes a plurality of codes corresponding to a plurality of display methods. By designating a specific code, a specific display position is designated. The display position is a position relative to the specific display area. Typically, the display position is specified by a relative position in the vertical direction and a relative position in the horizontal direction.

For example, in a case in which an upper left corner point of the external image is positioned to match an upper left corner point of the display area, a code 0 is designated. In that case, for example, the external image is displayed in an upper left corner region in the display area. The size may also be designated by designating the code. For example, in a case in which a code 3 is designated, the external image is displayed such that an upper side of the display area and an upper side of the external image match each other. In that case, the size (scale) of the external image is adjusted such that the width in the horizontal direction (horizontal width) of the display area and the width in the horizontal (horizontal width) of the external image match each other.

Basically, the size of the external image is adjusted such that the entirety of the external image fits within the display area, that is, such that the external image does not protrude from the display area. A part (protruding portion) of the external image is cut by the designated code.

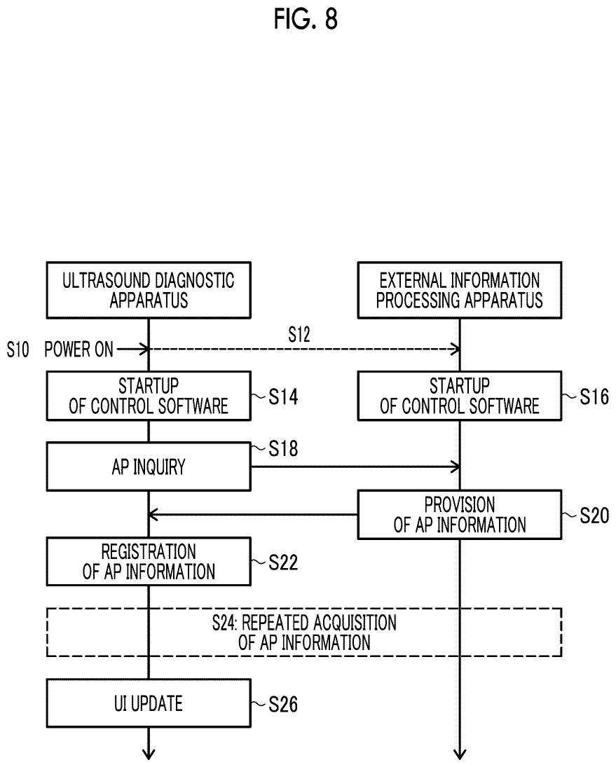

FIG. 8 shows an operation example during system startup. The left side of FIG. 8 shows an operation of the ultrasound diagnostic apparatus (particularly, an operation of the information processing unit), and the right side of FIG. 8 shows an operation of the external information processing apparatus (particularly, an operation of the information processing unit).

In S10, in a case in which the power of the ultrasound diagnostic apparatus is turned ON, the power of the external information processing apparatus is turned ON in S12. That is, two power sources operate in conjunction with each other. In S14, control software in the ultrasound diagnostic apparatus is started, and in S16, control software in the external information processing apparatus is started.

In S18, an inquiry of AP information is conducted from the ultrasound diagnostic apparatus to the external information processing apparatus. In S20, the AP information is provided from the external information processing apparatus to the ultrasound diagnostic apparatus. The external AP management table is generated based on the AP information. That is, in S22, the AP information is registered in the ultrasound diagnostic apparatus. In S24, in a case in which the external information processing apparatus is provided with two or more external APs, the acquisition of the AP information is repeated. In S26, an AP selection image (UI image) is generated based on the external AP management table.

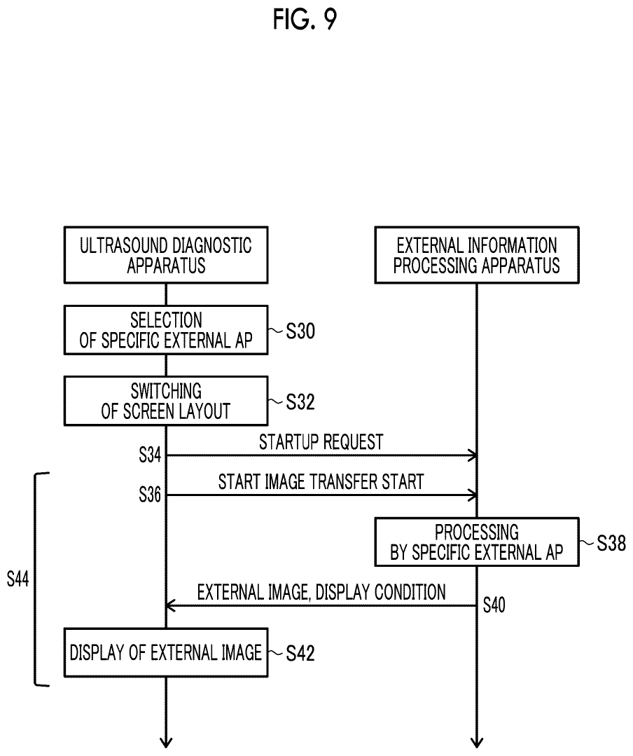

FIG. 9 shows a display operation example. The left side of FIG. 9 shows an operation of the ultrasound diagnostic apparatus (particularly, an operation of the information processing unit), and the right side of FIG. 9 shows an operation of the external information processing apparatus (particularly, an operation of the information processing unit).

In S30, a specific external AP is selected by the user. In S32, a specific screen layout corresponding to the specific external AP is selected. In that case, for example, by referring to the display format in the external AP management table, the specific screen layout is selected. At a point in time when the display condition is acquired in subsequent S40, the specific screen layout may be selected based on the display condition.

In S34, the startup of the specific external AP is requested from the ultrasound diagnostic apparatus to the external information processing apparatus. In S36, the transfer of the ultrasound image from the ultrasound diagnostic apparatus to the external information processing apparatus is started. In S38, the ultrasound image is processed by the specific external AP. Consequently, an external image representing the processing result is generated. In S40, the external image and the display condition are transferred from the external information processing apparatus to the ultrasound diagnostic apparatus. The display condition is generated by the specific external AP. The display condition may be generated by another module in the external information processing apparatus.

In S42, the actual display position (display coordinates) for displaying the external image is calculated, and the actual display size of the external image is calculated, based on the display condition on the premise of the selected specific screen layout. The external image having an actual display size is disposed at the actual display position in the specific display area on the display coordinate system. Consequently, a composite image including the external image is generated, and the composite image is displayed on the screen. S44 indicates the repeated operation in a case in which the external image is a moving image, that is, the repeated generation and display of the composite image.

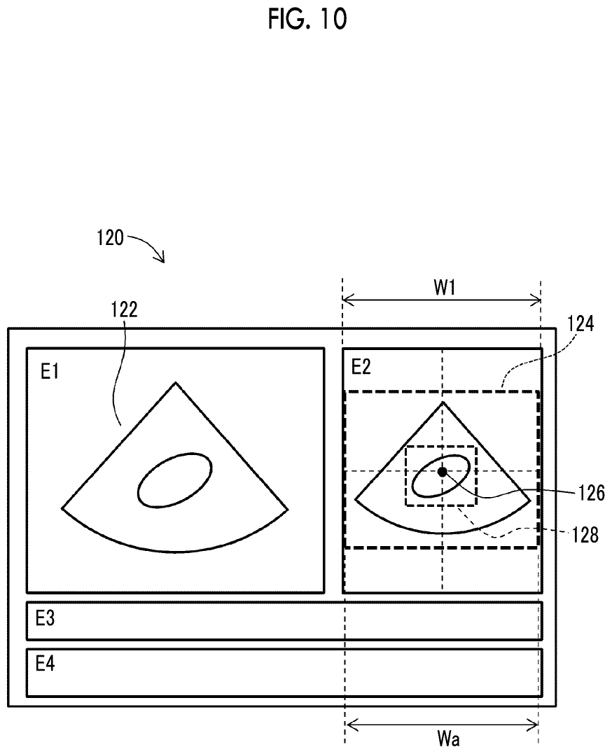

FIG. 10 shows a first display example. A composite image 120 generated in accordance with a specific screen layout is displayed on the screen. The specific screen layout includes the plurality of display areas E1 to E4. The display area E1 is an image display area, and the ultrasound image is displayed in the display area E1. The ultrasound image is, for example, a tomographic image that changes in real time, that is, a moving image.

The display area E2 is an image display area, and an external image 124 is displayed in the display area E2. In the shown example, the external image 124 is displayed such that a center point of the external image 124 matches a center point 126 of the display area E2. The horizontal width W1 of the display area E2 matches a horizontal width Wa of the external image 124. The external image 124 has a rectangular mark 128 surrounding the detected lesion part. In the shown example, the external image 124 is also a tomographic image that changes in real time, that is, a moving image. Obviously, a still image may be displayed as the external image.

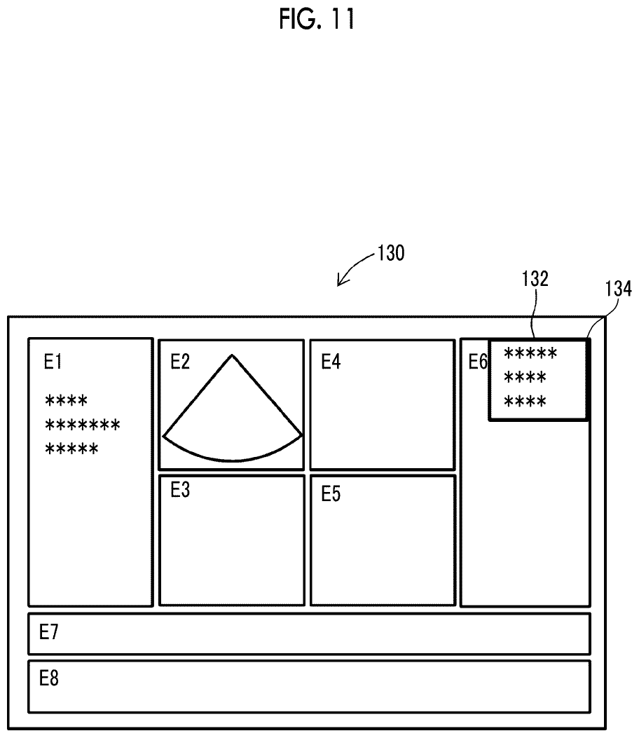

FIG. 11 shows a second display example. A composite image 130 generated in accordance with a specific screen layout is displayed on the screen. The specific screen layout includes a plurality of display areas E1 to E8. The display area E2 is an image display area, and the ultrasound image is displayed in the display area E2.

The display area E6 is an information display area, and an external image 132 is displayed in the display area E6. In the shown example, an upper right corner point of the external image 132 matches an upper right corner point 134 of the display area E6. The external image 132 includes a text image.

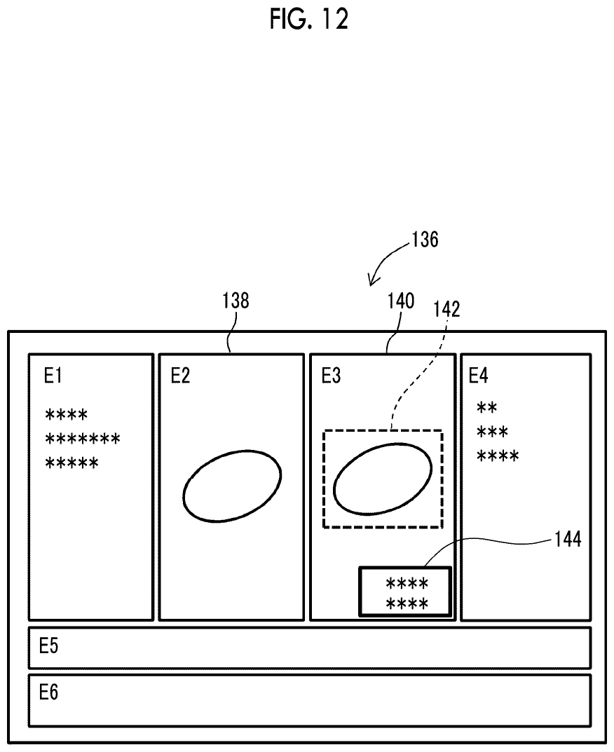

In FIG. 12, a composite image 136 generated in accordance with a specific screen layout is displayed. The specific screen layout includes the plurality of display areas E1 to E6. The display area E2 is an image display area, and the ultrasound image is displayed in the display area E2.

The display area E3 is also an image display area, and a first external image 140 is displayed in the display area E3. The first external image 140 includes a rectangular mark 142 surrounding the detected lesion part. In addition, a second external image 144 is also displayed in the display area E3. Specifically, the second external image 144 is superimposed and displayed to cover a lower right corner part of the first external image. In a case in which important information is hidden in displaying the second external image 144, the actual display position of the second external image may be automatically changed. Alternatively, the second external image 144 may be displayed semi-transparently.

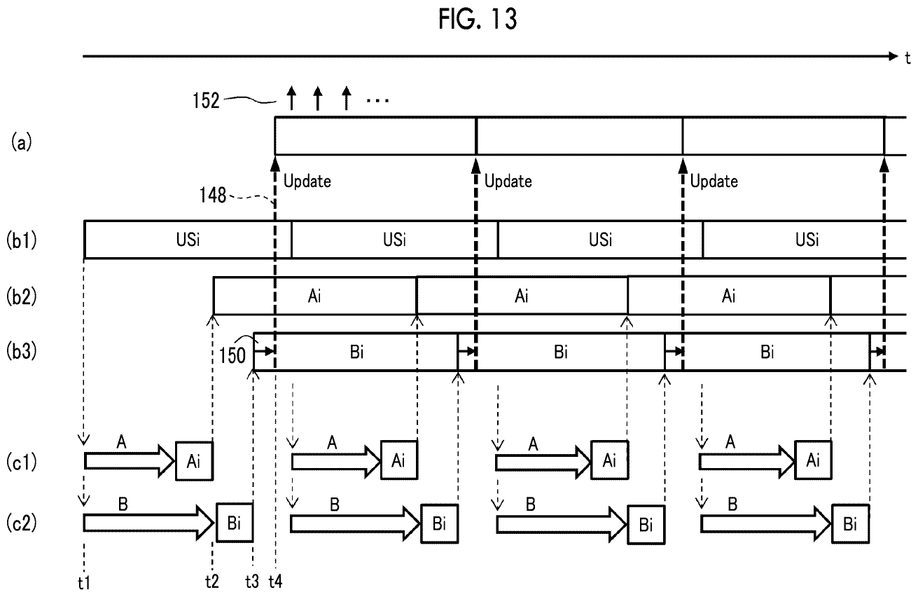

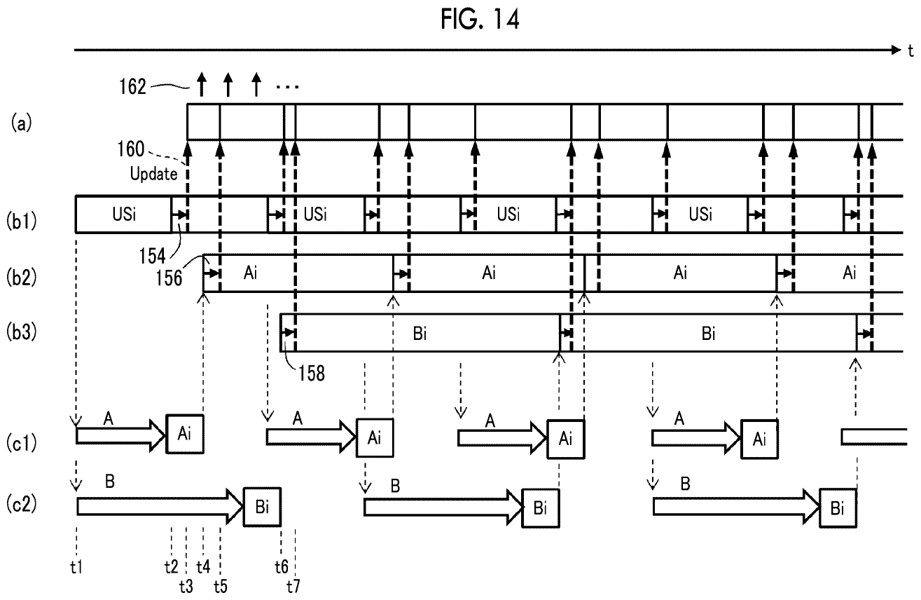

Next, display update control will be described by using FIGS. 13 and 14. FIG. 13 shows an operation example in a case in which the synchronous mode is selected, and FIG. 14 shows an operation example in a case in which the asynchronous mode is selected.

In FIG. 13, (a) indicates the storage of the composite image in a secondary buffer memory in the ultrasound diagnostic apparatus. (b1) indicates the storage of an ultrasound image USi in a primary buffer memory in the ultrasound diagnostic apparatus. (b2) indicates the storage of a first external image Ai in the primary buffer memory, and (b3) indicates the storage of a second external image Bi in the primary buffer memory. (c1) indicates processing A of a first external AP and the generation of the first external image Ai by the external information processing apparatus. (c2) indicates processing B of a second external AP and the generation of the second external image Bi by the external information processing apparatus.

At timing t1, the processing A and B of the ultrasound image USi is started in the first external AP and the second external AP. At timing t2, the first external image Ai is transferred to the primary buffer memory. At timing t3, the second external image Bi is transferred to the primary buffer memory.

After that, at timing t4 after the completion of the storage of the second external image Bi (refer to reference numeral 150), the ultrasound image USi, the first external image Ai, and the second external image Bi are collectively transferred from the primary buffer memory to the secondary buffer memory (refer to reference numeral 148). Consequently, the entire composite image on the secondary buffer memory is updated. As indicated by reference numeral 152, the composite image read from the secondary buffer memory is displayed on the display at a constant display rate.

As described above, in a case in which the synchronous mode is selected, a plurality of main images constituting the composite image are simultaneously updated. In particular, in a case in which the first external image Ai and the second external image Bi are generated in parallel by the external information processing apparatus, the two external images Ai and Bi to be displayed are updated together with the later generation timing as a reference. For example, in a case in which a temporal relationship between the content of the first external image Ai and the second external image Bi is emphasized, the synchronous mode is selected.

In FIGS. 14, (a) to (c2) are as described above. In FIG. 14, at timing t1, the processing A and B of the ultrasound image USi is started in the first external AP and the second external AP. At timing t2, the ultrasound image USi is updated, and at timing t3 after the completion of the update (refer to reference numeral 154), the new ultrasound image is transferred from the primary buffer memory to the secondary buffer memory. Reference numeral 160 indicates partial update of the composite image.

At timing t4, the first external image Ai is transferred to the primary buffer memory. At timing t5 after the completion of the transfer (refer to reference numeral 156), the first external image Ai is transferred from the primary buffer memory to the secondary buffer memory. At that time, the composite image on the secondary buffer memory is partially updated.

At timing t6, the second external image Bi is transferred to the primary buffer memory. At timing t7 after the completion of the transfer (refer to reference numeral 158), the second external image Bi is transferred from the primary buffer memory to the secondary buffer memory. At that time, the composite image on the secondary buffer memory is partially updated.

Immediately before timing t7, the ultrasound image USi on the primary buffer memory is updated, and after the update, the new ultrasound image USi is transferred from the primary buffer memory to the secondary buffer memory. As indicated by reference numeral 162, the composite image read from the secondary buffer memory is displayed on the display at a constant display rate.

As described above, in a case in which the asynchronous mode is selected, a plurality of images constituting the composite image are independently updated. In particular, the first external image Ai and the second external image Bi generated by the external information processing apparatus are independently and promptly updated. For example, in a case in which the temporal relationship between the content of the first external image Ai and the second external image Bi does not matter, and real-time display is emphasized, the asynchronous mode is selected.

In a case in which the two external APs designate two different modes, the mode may be selected by the user, or any one of the modes may be preferentially selected.

In the ultrasound diagnostic system according to the embodiment, the specific screen layout forms a common foundation for a designation of a printing condition by the external information processing apparatus and for display processing by the ultrasound diagnostic apparatus. That is, the display condition is designated by the external information processing apparatus on the premise of the specific screen layout. Meanwhile, the actual display position for displaying the external image is calculated by the ultrasound diagnostic apparatus in accordance with the display condition on the premise of the specific screen layout. With such a mechanism, in a case in which the external image is displayed on the display of the ultrasound diagnostic apparatus, it is possible to rationalize the cooperation between the external information processing apparatus and the ultrasound diagnostic apparatus. Further, with the ultrasound diagnostic system according to the embodiment, the update condition can be designated by the external AP, so that update control suitable for the function or the property of the external image is applied.

What is claimed is:

1. An ultrasound diagnostic apparatus comprising:
a processor; and
a display,
wherein the processor is configured to:
select a specific screen layout from among a plurality of screen layouts;
send an ultrasound image generated through transmission and reception of ultrasound waves to an external information processing apparatus;
receive an external image, which is an image generated through processing of the ultrasound image, and a display condition, which is used to display the external image and which includes a designation of a specific display area in the specific screen layout, from the external information processing apparatus; and
calculate an actual display position for displaying the external image based on the specific screen layout and the display condition to generate a composite image including the external image as an image to be displayed on the display.

2. The ultrasound diagnostic apparatus according to claim 1,
wherein the display condition further includes a designation of a display position in the specific display area, and
the display position is a position relative to the specific display area.

3. The ultrasound diagnostic apparatus according to claim 1,
wherein the display condition further includes a designation of a display size of the external image,
the display size is a size relative to the specific display area, and
the processor is configured to calculate an actual display size of the external image based on the designation of the display size.

4. The ultrasound diagnostic apparatus according to claim 1,
wherein the display condition further includes a direct or indirect designation of the specific screen layout, and
the processor is configured to select the specific screen layout based on the direct or indirect designation of the specific screen layout.

5. The ultrasound diagnostic apparatus according to claim 1,
wherein the display condition further includes an update condition, and
the processor is configured to control an update timing of the external image displayed on the display in accordance with the update condition.

6. The ultrasound diagnostic apparatus according to claim 5,
wherein the processor is configured to:
in a case in which a synchronous mode is designated as the update condition, and a plurality of the external images are generated through a plurality of image processing operations by the external information processing apparatus, simultaneously update the plurality of external images displayed on the display; and
in a case in which an asynchronous mode is designated as the update condition, and the plurality of external images are generated through the plurality of image processing operations by the external information processing apparatus, independently update the plurality of external images displayed on the display.

7. An ultrasound diagnostic system comprising:
an ultrasound diagnostic apparatus including a processor and a display; and
an external information processing apparatus connected to the ultrasound diagnostic apparatus,
wherein the processor is configured to:
select a specific screen layout from among a plurality of screen layouts;
send an ultrasound image generated through transmission and reception of ultrasound waves to the external information processing apparatus;
receive an external image, which is an image generated through processing of the ultrasound image, and a display condition, which is used to display the external image and which includes a designation of a specific display area in the specific screen layout, from the external information processing apparatus; and
calculate an actual display position for displaying the external image based on the specific screen layout and the display condition to generate a composite image including the external image as an image to be displayed on the display.

8. A non-transitory storage medium storing a program executed by an ultrasound diagnostic apparatus including a display, the program comprising:
a function of selecting a specific screen layout from among a plurality of screen layouts;
a function of sending an ultrasound image generated through transmission and reception of ultrasound waves to an external information processing apparatus;
a function of receiving an external image, which is an image generated through processing of the ultrasound image, and a display condition, which is used to display the external image and which includes a designation of a specific display area in the specific screen layout, from the external information processing apparatus; and
a function of calculating an actual display position for displaying the external image based on the specific screen layout and the display condition to generate a composite image including the external image as an image to be displayed on the display.
